(19) United States
(12) Patent Application Publication
(10) Pub. No.: US 2024/0000459 A1
(43) Pub. Date: Jan. 4, 2024

(54) EMBOLISATION SYSTEM FOR PROMOTING CLOT FORMATION

(71) Applicant: Clearstream Technologies Limited, Enniscorthy, County Wexford (IE)

(72) Inventors: Pat Byrne, Enniscorthy (IE); Seán Wall, Enniscorthy (IE); Stephen Sheridan, Enniscorthy (IE)

(73) Assignee: Clearstream Technologies Limited, Enniscorthy, County Wexford (IE)

(21) Appl. No.: 18/255,617

(22) PCT Filed: Dec. 4, 2020
(86) PCT No.: PCT/EP2020/084654

§ 371 Date: Jun. 2, 2023

(51) Int. Cl. A61B 17/12

(52) U.S. Cl. CPC A61B 17/12145; A61B 17/12109; A61B 2017/00526

(57) ABSTRACT

An embolisation device (300, 500) for promoting clot formation in a body lumen comprising two or more linearly connected sections (305, 505), each section comprising one or more bristle segments (310, 510) comprising a core and a plurality of flexible bristles (330, 530) extending at least radially outwardly from the core, the flexible bristles having a collapsed delivery configuration and an expanded deployed configuration in which the bristles extend at least radially outwardly from the core to anchor the bristle segment in a bodily lumen, wherein: each pair of adjacent sections are connected via a respective connecting mechanism; and when the bristle segments are in the expanded deployed configuration, each respective connecting mechanism is selectively changeable from a first configuration attaching the pair of adjacent sections, to a second configuration detaching the pair of adjacent sections.

### TECHNICAL FIELD

The present disclosure relates to an embolisation bristle section for promoting clot formation in a bodily lumen, having a collapsed delivery configuration and an expanded deployed configuration for anchoring the embolisation device in the bodily lumen. The present disclosure also relates to an embolisation device comprising a plurality of bristle sections, a method of manufacturing the bristle section and embolisation device, and a kit of parts for making the embolisation device.

### BACKGROUND

Embolisation devices may be deployed in the vasculature at a particular location by a medical practitioner so as to promote blood clot formation and ultimately occlude the blood vessel. Typically, an embolisation device may be pushed through a guide (delivery) catheter in a distal direction using a delivery wire until a point of deployment within a bodily lumen is reached. Once the device reaches the required point of deployment, the device is deployed from the guide catheter.

Different sized embolisation devices (both in diameter and length) may be desired for different blood vessel sizes and shapes. Embolisation devices of a standard shape and size may not be able to effectively occlude blood vessels having a particular shape.

Accordingly, there is a need to provide embolisation systems which may be easily and effectively deployed in a diverse range of blood vessels.

### SUMMARY

According to a first aspect, there is provided an embolisation device for promoting clot formation in a body lumen comprising two or more linearly connected sections, each section comprising one or more bristle segments comprising a core and a plurality of flexible bristles extending radially outwardly from the core, the flexible bristles having a collapsed delivery configuration and an expanded deployed configuration in which the bristles extend at least radially outwardly from the core to anchor the bristle segment in a body lumen, wherein: each pair of adjacent sections are connected via a respective connecting mechanism; and each respective connecting mechanism is selectively changeable from a first configuration attaching the pair of adjacent sections, to a second configuration detaching the pair of adjacent sections, when the bristle segments are in the expanded deployed configuration. The connecting mechanisms allow the positioning and deployment of the bristle sections to be highly controllable.

At least one connecting mechanism may be movable from its first configuration to its second configuration.

At least one connecting mechanism may be formed by an elongate element slidably received by respective receiving elements of a pair of adjacent sections, such that the elongate element is slidable by a predetermined distance between the first and second configurations, and wherein in the second configuration the elongate element detaches from one of the receiving elements to detach the pair of adjacent sections. Such a configuration allows a user to determine when a pair of sections has been detached once the elongate element has been retracted by the predetermined distance, allowing for a controlled detach mechanism in the lumen for each section.

The embolisation device may have a longitudinal axis and the receiving elements may each comprise respective interlocking features, wherein in the first configuration the respective interlocking features interlock with one another in a lateral direction to inhibit relative displacement in the longitudinal direction between the pair of adjacent sections, and the elongate element passes through both interlocking features in a longitudinal direction to inhibit relative lateral movement between the pair of adjacent sections. As the longitudinal separation between the adjacent sections is inhibited by the interlocking features of the receiving elements, damage to the connecting mechanism by longitudinal forces on the embolisation device may be prevented. In the second configuration, as lateral movement is no longer prevented by the connecting mechanism (due to it being extracted from the receiving element), the sections easily detach.

The respective interlocking features may each comprise a lip and a lateral recess, wherein in the first configuration the respective lips may be received by the lateral recess of the other respective interlocking feature, and the elongate element may pass through both lips in a longitudinal direction to inhibit relative lateral movement between the pair of adjacent sections.

A first of the pair of receiving elements may comprise a base longitudinally separated from the lip by the recess, the base having a hole extending therethrough having a longitudinally extending section and a laterally extending section, wherein in the first configuration the elongate element passes through the lip of the first receiving element, the lip of the other receiving element, the longitudinally extending section of the hole and the laterally extending section of the hole. The provision of the longitudinally and laterally extending sections of the hole increase the friction between the receiving element and the elongate element, which may reduce the risk of the elongate element coming loose from the receiving element prematurely.

At least two of the connecting mechanisms may be formed by the same elongate element received by a plurality of pairs of receiving elements such that the elongate element is slidable by a plurality of predetermined distances to detach a pair of sections at each predetermined distance. Such a configuration allows multiple sections to be deployed by manipulating a single elongate element, meaning that a mechanism configured to deploy the sections of the embolisation device can be simplified.

At least one connecting mechanism may formed by an electrolytic element attaching a respective pair of adjacent sections, the electrolytic element being electrically connected to a proximal end of the embolisation device, and operable to disintegrate by electrolysis in the body lumen to detach the respective pair of adjacent sections by applying an electric current to the electrolytic element, at a current amplitude, a voltage and for a duration of time, such that the electrical energy supplied to the electrolytic element is above a disintegration energy of the electrolytic element. Such a configuration allows a user to be able to detach the bristle sections by applying a positive current at a particular current amplitude and voltage for a given duration of time, allowing for a controlled detach mechanism. The electric current may be a positive current.

The electrical connection may be formed by an electrical wire extending between the proximal end of the embolisation device to the electrolytic element. The electrical wire may be electrically isolated from the cores and/or bristles of the embolisation device, to inhibit electrolytic corrosion of the cores/bristles.

At least two connecting mechanisms may each be formed by such an electrolytic element, wherein the disintegration energy of each electrolytic element is different. The different disintegration energies may allow the sections to be deployed independently in a highly controllable manner.

According to a second aspect, there is provided an embolisation bristle section for promoting clot formation in a body lumen comprising one or more linearly connected bristle segments, each bristle segment comprising a core and a plurality of flexible bristles extending radially outwardly from the core, the flexible bristles having a collapsed delivery configuration and an expanded deployed configuration in which the bristles extend at least radially outwardly from the core to anchor the device in a lumen, wherein: the bristle section further comprises a receiving element configured to slidably receive a connecting mechanism for connecting the bristle section to an adjacent bristle section, wherein, the receiving element is configured to allow the connecting mechanism to slide between a first configuration for attaching the bristle section and the adjacent bristle section, and a second configuration for detaching the bristle segment from the adjacent bristle section. A user may readily attach multiple bristle sections together to create an embolisation device of a custom length.

According to a third aspect, there is provided an embolisation delivery system for delivering an embolisation device according to the first aspect, comprising: a delivery catheter for containing the embolisation device and having a distal delivery end; a delivery element for delivering the embolisation device from the distal end of the delivery catheter; an actuator for changing the one or more connecting mechanisms from the first configuration to the second configuration; and one or more user interfaces for operating the delivery element and the actuator. The one or more user interfaces allow a user to control deployment of a bristle section within a lumen, and to control detachment of the bristle section within the lumen.

When one or more of the connecting mechanisms comprise one or more elongate elements slidable between first and second configurations, the actuator may comprise a retracting element operable by a user to slide the one or more connecting mechanisms from the first to the second configuration. When one or more of the connecting mechanisms comprise one or more electrolytic elements changeable from the first configurations to the second configurations by application of a positive current to the one or more electrolytic elements, the actuator may be operable by a user to apply a positive current to the one or more connecting mechanisms for detachment.

When one or more of the connecting mechanisms are elongate elements slidable between the first and second configurations, the retracting element may comprise a feedback mechanism for indicating to the user that the one or more connecting mechanisms have slid a sufficient distance to cause detachment.

When one or more of the connecting mechanisms are electrolytic elements, the actuator may be operable to apply a variable positive current and voltage selectable by the user via the user interface. The user interface allows the user to select which electrolytic elements to be actuated to detach the respective bristle sections.

According to a fourth aspect, there is provided a method of manufacturing an embolisation device for promoting clot formation in a body lumen comprising: providing a plurality of bristle sections each comprising one or more bristle segments, each bristle segment comprising a core and a plurality of flexible bristles extending radially outwardly from the core, the flexible bristles having a collapsed delivery configuration and an expanded deployed configuration in which the bristles extend at least radially outwardly from the core to anchor the bristle section in a lumen; linearly connecting adjacent pairs of the bristle sections via one or more connecting mechanisms, each connecting mechanism being operable to change from a first configuration attaching a pair of adjacent sections, to a second configuration detaching the pair of adjacent sections, when the bristle segments are in the expanded deployed configuration.

According to a fifth aspect, there is provided a method of manufacturing an embolisation bristle section for promoting clot formation in a body lumen, comprising: providing one or more bristle segments, each bristle segment comprising a core and a plurality of flexible bristles extending radially outwardly from the core, the flexible bristles having a collapsed delivery configuration and an expanded deployed configuration in which the bristles extend at least radially outwardly from the core to anchor the device in a lumen; providing a receiving element configured to slidably receive an elongate element for connecting the bristle section to an adjacent bristle section, wherein the receiving element is configured to allow the elongate element to slide between a first configuration attaching the bristle section and the adjacent bristle section, and a second configuration detaching the bristle segment from the adjacent bristle section.

According to a sixth aspect, there is provided a kit of parts for making an embolisation device, comprising: two or more sections, each section comprising: one or more bristle segments comprising a core and a plurality of flexible bristles extending radially outwardly from the core, the flexible bristles having a collapsed delivery configuration and an expanded deployed configuration in which the bristles extend at least radially outwardly from the core to anchor the bristle segment in a lumen; and one or more receiving elements configured to slidably receive an elongate element for connecting the bristle section to an adjacent bristle section, wherein the receiving element is configured to allow the elongate element to slide between a first configuration attaching the bristle section and the adjacent bristle section, and a second configuration detaching the bristle segment from the adjacent bristle section; the kit of parts further comprising one or more elongate elements for connecting the bristle sections. The kit of parts may be usable to construct a custom embolisation device which is adapted to a particular vessel size or shape.

### BRIEF DESCRIPTION OF THE DRAWINGS

To enable better understanding of the present disclosure, and to show how the same may be carried into effect, reference will now be made, by way of example only, to the accompanying schematic drawings, in which:

FIG. 1A shows a side view of an embolisation bristle segment in an unconstrained configuration according to one or more embodiments shown and described herein;

FIG. 1B shows a side view of a plurality of bristle segments of a bristle section according to one or more embodiments shown and described herein;

FIG. 1C shows a joint connecting two bristle segments according to one or more embodiments shown and described herein;

FIG. 1D shows a joint connecting two bristle segments according to one or more embodiments shown and described herein;

FIG. 1E shows a joint connecting two bristle segments according to one or more embodiments shown and described herein;

FIG. 2 shows an embolisation delivery system according to one or more examples;

FIG. 3A shows a side view embolisation device in a first configuration according to one or more embodiments shown and described herein

FIG. 3B shows a side view of an embolisation device in a second configuration according to one or more embodiments shown and described herein;

FIG. 4A shows a side view of pair of receiving elements for an embolisation bristle section according to one or more embodiments shown and described herein;

FIG. 4B shows a side view of a pair of receiving elements for an embolisation bristle section according to one or more embodiments shown and described herein;

FIG. 5A shows a side view of another embolisation device in a first configuration according to one or more embodiments shown and described herein;

FIG. 5B shows a side view of the embolisation device shown in FIG. 5A in a second configuration according to one or more embodiments shown and described herein;

FIG. 6A shows an embolisation system for delivering an embolisation device according to one or more embodiments shown and described herein;

FIG. 6B shows the embolisation system for delivering an embolisation device of FIG. 6A with a bristle section in a deployed configuration according to one or more embodiments shown and described herein;

FIG. 6C shows the embolisation system for delivering an embolisation device of FIG. 6A with a bristle section deployed and detached according to one or more embodiments shown and described herein;

FIG. 7A shows an embolisation system for delivering an embolisation device according to one or more embodiments shown and described herein;

FIG. 7B shows the embolisation system for delivering an embolisation device of FIG. 7A with a bristle section in a deployed configuration according to one or more embodiments shown and described herein; and

FIG. 7C shows the embolisation system for delivering an embolisation device of FIG. 7A with a bristle section deployed and detached according to one or more embodiments shown and described herein.

### DETAILED DESCRIPTION

Throughout this disclosure the term ‘embolisation device’ may refer to a device which may be permanently or semi-permanently implanted in a bodily lumen to promote occlusion of the bodily lumen to embolise the bodily lumen. Accordingly, the ‘embolisation device’ may be configured to be disposed within the bodily lumen for a period of time, such as a number of days, or disposed in the lumen indefinitely, to promote occlusion of the lumen. To this end, the ‘embolisation device’ may be configured to be selectively detached from a delivery element so that it may be implanted in the bodily lumen in isolation.

Throughout this disclosure the term ‘bodily lumen’ may refer to the inside space within a tubular structure of the human or animal body. The ‘bodily lumen’ may be, for example, an artery or vein.

Throughout this disclosure the term ‘collapsed delivery configuration’ of an element may refer to a configuration of the element which has a smaller radial extent than an expanded deployed configuration of the element.

Throughout this disclosure the term ‘to anchor’ may refer to partly or fully securing an element in a position.

The embolisation devices disclosed herein comprise bristle segments having a core and a plurality of flexible bristles extending radially outwardly of the core. The core may be a stem. The term ‘stem’ may refer to an elongate element which extends longitudinally along the length of the embolisation device or bristle segment to act as a backbone for the device or segment, and has a significantly smaller radial extent than the further elements of the embolisation device (for example, the plurality of flexible bristles). The stem may extend along substantially the whole longitudinal extent of the plurality of flexible bristles of the device or segment (e.g. when the embolisation device is in an unrestrained configuration, collapsed delivery configuration and/or expanded deployed configuration). The stem may extend along substantially the whole length of the embolisation device. The bristle segments of each bristle section may be formed on a single core or may be formed on separate connected cores.

In any of the examples described herein, the term ‘bristle’ may refer to an elongate strand of material formed substantially a single piece. The ‘bristle’ may be a resilient bristle. The resilient bristle may be biased towards a particular curvature.

The embolisation devices of the present disclosure comprise a plurality of linearly connected sections, each section comprising one or more bristle segments. Where a section comprises a plurality of bristle segments, these bristle segments may be linearly connected. For example, adjacent bristle segments may be separated bristle segments formed on the same core or may be linearly connected via a joint, such as an articulating joint.

Throughout this disclosure, the term ‘radially outwardly’ does not exclude the element additionally extending in the longitudinal direction of the device. For example, the plurality of flexible bristles may extend radially outwardly and longitudinally from the stem.

Through this disclosure, the term “radial profile” may refer to a radial extent in a particular direction radially outwardly from the stem of the embolisation device. For example, the embolisation device has a lower radial extent in the collapsed delivery configuration than in the expanded deployed configuration.

Throughout this disclosure, the term “bristle segment” may refer to a group of bristles wherein the spacing between adjacent bristles is less than a predetermined distance. When two bristle segments are “spaced apart”, the spacing between the bristle segments (i.e. the spacing between the most distal bristle of the first segment and the most proximal bristle of the second segment) is greater than the spacing between adjacent bristles within at least one of the bristle segments.

The plurality of flexible bristles may have a collapsed configuration in the collapsed delivery configuration. The plurality of flexible bristles may have an expanded configuration in the expanded deployed configuration. The plurality of bristles may extend radially outwardly from the core or stem in a plurality of circumferential directions about the core or stem.

In the expanded configuration, the plurality of flexible bristles may be configured to anchor the device in the bodily lumen. The plurality of flexible bristles may be configured to provide substantially all of the anchoring force for the embolisation device or the bristle segment in the bodily lumen.

In the expanded configuration, the plurality of flexible bristles may be configured to contact the bodily lumen.

The device adopts the collapsed delivery configuration when the device is positioned inside the delivery catheter. More particularly, in the collapsed delivery configuration, the plurality of flexible bristles extending outwardly from the core or stem have a radial extent which is less than the radial extent of the bristles in the expanded deployed configuration of the element.

The embolisation devices disclosed herein may comprise one or more flow restrictors disposed on the core or stem of one or more of the bristle segments. The flow restrictor may be placed at any point on the bristle segments, for example longitudinally within the bristles of the bristle segment, longitudinally outside of, and/or spaced apart from the bristles of the bristle segment.

The bristles may be made of a flexible or resiliently deformable material such as stainless steel, Elgiloy or Nitinol. Other suitable materials may also be used, such as any suitable polymer or any other shape memory metal or metal alloy. The flow restrictor may be a thin film membrane made of a self-expanding material such as a polymer, stainless steel or Nitinol. The core or stem may be made of stainless steel or other suitable material and may comprise a twisted wire from which the bristles extend and on which the flow restrictor is mounted. The stem may alternatively comprise a hollow tube wherein the walls of the hollow tube hold the radially extending bristles in place. For example, the tube may be formed from a shrinkable material. Alternatively or additionally, the bristles may be held by the stem using other means such as adhesives.

The diameter of an individual flexible bristle may range from 0.036 mm (0.0014 inches) to 0.053 mm (0.0021 inches). For example, the diameter of an individual flexible bristle may be 0.0381 mm (0.0015 inches), 0.0445 mm (0.00175 inches) or 0.0508 mm (0.002 inches). The average radial diameter of the radial profile formed by the expanded flexible bristles may range from 5 mm to 30 mm.

A flow restrictor may be a membrane made from thin film Nitinol, thin film PTFE, a thin film elastomer such as polyurethane or any other type of suitable biocompatible material. The membrane may have a thickness of 4 μm to 35 μm and a radial diameter of 5 mm to 20 mm. For example, the diameter of the membrane may be 6.5 mm, 9 mm or 16 mm. Furthermore, the membrane may be non-permeable or semi-permeable.

In examples where a connecting mechanism comprises an elongate element, suitable materials include biocompatible metals, for example stainless steel, nitinol, Elgiloy including radiopaque metals such as platinum, Pt/Ir alloys, tantalum or gold. Alternatively, polymeric materials may be used, such as ultra-high-molecular-weight polyethylene (e.g. dyneema), aramids (such as technora), or nylons (such as an extruded or film cast mix of nylon materials like Nylon 12—e.g. polyamide 12 laurolactam family and variants thereof such as Grilamid L25, L20LF etc., or or Vestamid L series, or Rilsan, or other thermoplastic variants of Nylon 12 like Grilamid TR55—or Nylon 11 (e.g. Besvo or Besno) or Nylon 6 ( ). The polymeric materials may be coated or embedded with radiopaque material. The receiving elements which slidably receive the elongate element may be made of any biocompatible material, such as stainless steel, cobalt chrome, eligloy, polymeric materials such as polycarbonate, nylon (optionally with a pebax housing), PE, PTFE, PS, or composites thereof.

When the receiving elements and elongate elements are made of radiopaque material, a user may be able to directly verify whether the bristle sections are detached by radio imaging.

The electrolytic elements may be formed of platinum, stainless steel, nitinol and cobalt chromium. The disintegration energy required to cause the electrolytic element to disintegrate is determined by the duration of application of a direct positive current at a particular current amplitude and voltage. This parameter may therefore be varied between connecting mechanisms such that specific connecting mechanisms can be selectively disintegrated. For example, one electrolytic element may be configured to disintegrate after a current between 0.01 and 0.1 mA and a voltage between 8 to 10V is applied for a period of time, for example 30 seconds. Another electrolytic element may be configured to disintegrate after a current between 0.5-1.10 mA and 5 to 7V is applied for a period of time, for example 30 seconds. In embodiments where the electrolytic elements are connected by the same electrical connection, it will be appreciated that non-overlapping disintegration energies can be selected such that the electrolytic elements predictably disintegrate in order. For example, the materials of each electrolytic element, and the amount of material used, may determine how much electrical energy must be applied to each electrolytic element for it to disintegrate, and so these may be varied between the connections in order to controllably disintegrate the electrolytic elements.

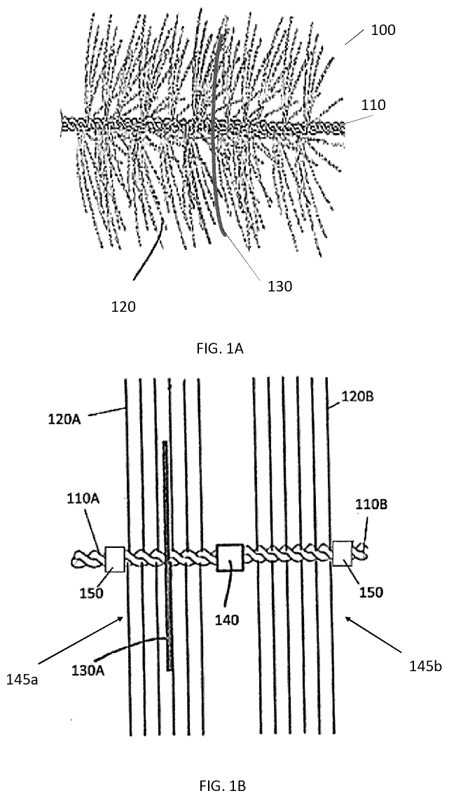

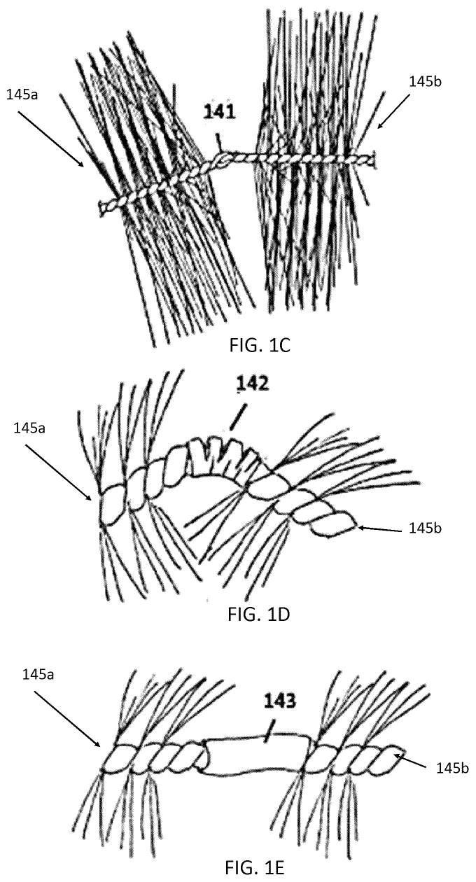

FIG. 1A shows an embolisation bristle segment 100 in an unconstrained configuration. The embolisation segment 100 comprises a core, and more specifically a stem, 110 and a plurality of bristles 120 extending generally (i.e. at least) radially outwardly from the stem 110. The embolisation segment may further comprise a flow restrictor 130, and more specifically a flow restricting membrane, mounted on the stem 110, although it will be apparent to the skilled person that this feature is optional. It will further be apparent to the skilled person that the position of the flow restrictor may vary, such as longitudinally within the bristles or adjacent the outermost bristles of the bristle segment or spaced apart from the outermost bristles.

The stem 110 illustrated comprises a twisted wire from which the bristles 120 extend, although as described above the stem may take other forms such as a hollow tube.

The embolisation bristle segment 100 may form part of a bristle section. The bristle segment may be connected to further bristle segments in the bristle section. For example, as shown in FIG. 1B, two bristle segments 145a, 145b may be connected via an interconnecting module 140. The interconnecting module 140 shown in FIG. 1B may be a crimping connector made of a flexible or inflexible material. Alternatively, bristle segments 145a, 145b may be connected by an articulating joint 141 such as that shown in FIG. 1C, a flexible joint such as the flexible tube 142 of FIG. 1D which may comprise slots to accommodate bending of the tube, or an inflexible joint 143 such as that shown in FIG. 1D. The joints may be made of a radiopaque material or may comprise a band of radiopaque material. Alternatively, the bristle segment 100 may be formed on the same stem as one or more further bristle segments. Alternatively or additionally, the bristle sections disclosed herein may comprise radiopaque markers 150. The radiopaque markers 150 (and where applicable the interconnecting module 140, when radiopaque), may assist a user in viewing when each bristle section is at the tip of the delivery catheter or has been deployed from the delivery catheter.

Each bristle section disclosed herein may comprise only one bristle segment or may comprise two or more bristle segments connected in any of the manners as described above.

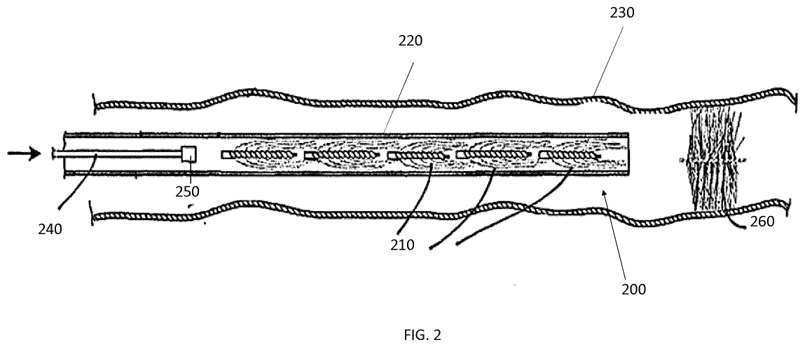

FIG. 2 shows an embolisation delivery system 200 inserted into a bodily lumen 230, containing a plurality of bristle segments 210. The delivery system 200 comprises a delivery catheter 220 and a delivery element 240 with a push feature 250. The push feature 250 preferably has a radial extent which is wider than the delivery element. The push feature 250 is configured to abut the most proximal bristle segment when the delivery element 240 is advance through the delivery catheter. The delivery element 240 is used to guide the bristle segments 210 through the delivery catheter 220 in a proximal direction towards a target site within the bodily lumen 230. In FIG. 2, the bristle segments 210 are shown in the collapsed delivery configuration.

In use, the bristle segments 210 are pushed through the delivery catheter 220 by the delivery element 240 (and specifically by the push feature 250 of the delivery element). The bristle segments 210 are then deployed from the distal end of the delivery catheter 220 to a target site of the bodily lumen 230. When a bristle segment 210 exits the distal end of the delivery catheter 220, the bristle segment moves to an expanded deployed configuration in which the bristles of the bristle segment engage the walls of the bodily lumen 230 to anchor the segment to the lumen. The bristle segment 260 is shown in the expanded deployed configuration. The bristle segments being separately deployable enables the bristle segments to be deployed in a wider variety of vasculature (as opposed to a single embolisation device comprising a chain of permanently attached bristle segments).

In the example shown in FIG. 2, the delivery system 200 is only able to push the bristle segments 210 in a distal direction, which reduces the amount of control a user has for deploying the bristle segments 210.

In alternative examples, the bristle segments are linearly attached to one another, for example via articulating joints or by them being formed on a single stem. The delivery element may connect to the most proximal bristle segment via a threaded connection, wherein the most proximal bristle segment comprises a male/female screw thread, and the distal end of the delivery element comprises a corresponding female/male screw thread. As a result, the delivery element may be used to both push the bristle segments through the delivery catheter and out of the distal end of the delivery catheter for deployment in the lumen, and also to pull the bristle segments back inside the delivery catheter (for example if it is discovered that the bristle segments have been positioned incorrectly within the bodily lumen). When the bristle segments have been correctly positioned, the delivery element is twisted until the screw threads detach.

Whilst such a configuration allows the delivery element to both deploy and recapture the bristle segments, the delivery element is only able to deliver the bristle segments in bulk — in other words, the delivery element cannot selectively deliver a sub-set of bristle segments at a time.

In light of the above drawbacks, there is disclosed herein an embolisation device comprising two or more linearly connected sections, each section comprising one or more bristle segments and being selectively detachable from the other bristle sections. The bristle segments in each bristle section may be of the form shown in FIG. 1A or any of the alternatives as described in relation to FIG. 1A. For bristle sections comprising a plurality of bristle segments, the constituent bristle segments belonging to a single bristle section may be linearly attached via non-articulating or articulating joints as described above, or may be discrete separated bristle segments formed on a single stem. Each pair of adjacent sections are connected via a respective connecting mechanism. When the bristle segments are in the expanded deployed configuration, each respective connecting mechanism is selectively changeable from a first configuration attaching the pair of adjacent sections, to a second configuration detaching the pair of adjacent sections. When such an embolisation device is attached to a delivery element configured to operably connect to the connecting mechanisms, the delivery element may be able to selectively detach certain bristle sections from the embolisation device. Therefore, in contrast to the examples described above, the bristle sections are suitable for being deployed separately within a bodily lumen but may also be recaptured after being expelled from the distal end of a delivery catheter. This allows for the positional correction of individual bristle sections whilst still allowing each bristle section to be deployed separately, increasing the controllability of the embolisation device.

It will be understood the skilled person that each bristle section may comprise a single bristle segment or any other number of bristle segments.

The connecting mechanisms may be changeable between the first and second configurations in a number of different ways. For example, the connecting mechanism may be movable between the first and second configurations. Alternatively, the connecting mechanisms may be configured to change between the first and second configurations via another mechanism, such as by electrolysis. Different types connecting mechanisms may be comprised on the same embolisation device, so long as they are operable to change between the configurations (i.e. connecting mechanisms are actuatable).

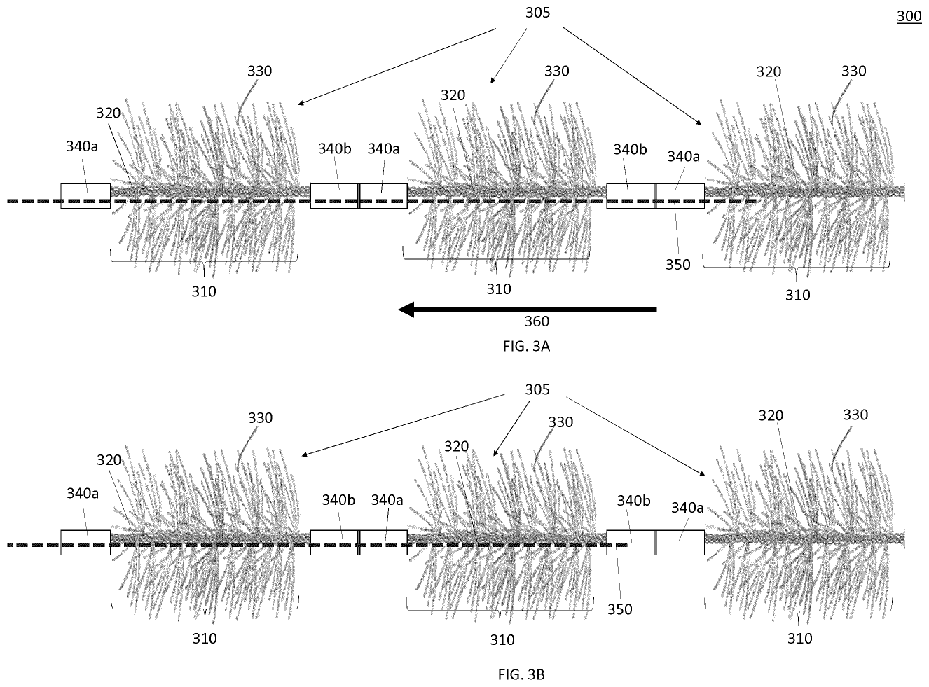

FIG. 3A shows one such embolisation device 300 in an unconstrained configuration. The embolisation device 300 comprises three bristle sections 305. In other examples, the embolisation device may comprise two bristle sections or more than three bristle sections. Each bristle section comprises a single bristle segment 310. In other examples, each bristle section may comprise a different number of bristle segments. For example, one bristle section may comprise a single bristle segment and another may comprise two or more bristle segments. One or more bristle sections may comprise a flow restrictor mounted on the stem as disclosed previously. As before, each bristle segment 310 comprises a core, and more specifically a stem 320 and a plurality of bristles 330 having a collapsed delivery configuration and an expanded deployed configuration. A proximal direction is indicated by the arrow 360.

The bristle sections 310 comprise receiving elements 340a, 340b, mounted on the stems 320, which slidably receive an elongate element 350. The elongate element 350 may be any suitable rod or wire which is configured to slidably fit in the elongate element 350. The rod or wire may have any suitable cross-sectional shape. The receiving elements 340a, 340b may be mounted on the stem by adhesive, welding, crimping, or by any other form of attachment. Adjacent pairs of receiving elements 340a, 340b, are connected to one another via the elongate element 350. For example, the receiving element 340b of the most proximal bristle section 305 is adjacent to the receiving element 340a of the distally adjacent bristle section 305. As the elongate element 350 is received by the adjacent receiving elements 340a, 340b, the pair of bristle sections are connected to one another by the elongate element 350. In one example, the receiving elements may each receive the elongate element 350 through a hole in a frictional fit. The elongate element 350 will inhibit lateral relative movement between the adjacent bristle sections 305 of the embolisation device 300 as it is received by receiving elements of adjacent bristle sections. Furthermore, the frictional fit inhibits relative longitudinal movement of the adjacent bristle sections.

It will be understood by the skilled person that the most proximal receiving element 340a in the embolisation device 300 is optional. In some examples, the most proximal end of the embolisation device 300 may have a connecting module for connecting to a delivery element such as the male or female screw thread described above. Alternatively, the most proximal end of the embolisation device 300 may comprise a receiving element 340a as shown in FIG. 3A, such that the embolisation device 300 is configured to attach to a similar receiving element on a distal end of a delivery element.

Similar to the system shown in FIG. 2, the embolisation device 300 may be placed in a delivery catheter for delivery to a target site in a bodily lumen. A delivery element may be connected to the proximal end of the embolisation device 300, for example via a screw thread mechanism or other, or by respective receiving elements slidably receiving the elongate element to attach the most proximal bristle section of the embolisation device 300 to the delivery element. The delivery element, connected to the proximal end of the embolisation device 300, is used to guide the bristle sections 305 through the delivery catheter in a proximal direction towards a target site within the bodily lumen.

The bristle sections may then be deployed from the distal end of the delivery catheter to a target site of the bodily lumen. When a bristle section 305 exits the distal end of the delivery catheter (i.e. the most distal bristle section), the bristle section 305 moves from a collapsed delivery configuration within the delivery catheter to an expanded deployed configuration in which the bristles of the bristle section (i.e. the bristles 330 of the one or more bristle segments 310) engage the walls of the bodily lumen to anchor the bristle section 305 to the lumen. When the bristle section 305 is positioned within the bodily lumen in the expanded deployed configuration (i.e. when it has exited the delivery catheter), it is initially still attached to the next most proximal bristle section via elongate element 350, and relative longitudinal and lateral movement between these bristle sections is still inhibited. As such, if it is determined that the most distal bristle section is incorrectly positioned, the delivery element may be moved in a proximal direction relative to the delivery catheter so that the bristle sections 305 all move proximally (due to the chain of connections between the delivery element and the linearly connected bristle sections), such that the distal bristle section in the bodily lumen is recaptured by the delivery catheter. The bristle section may then be delivered from the delivery catheter to a new position within bodily lumen.

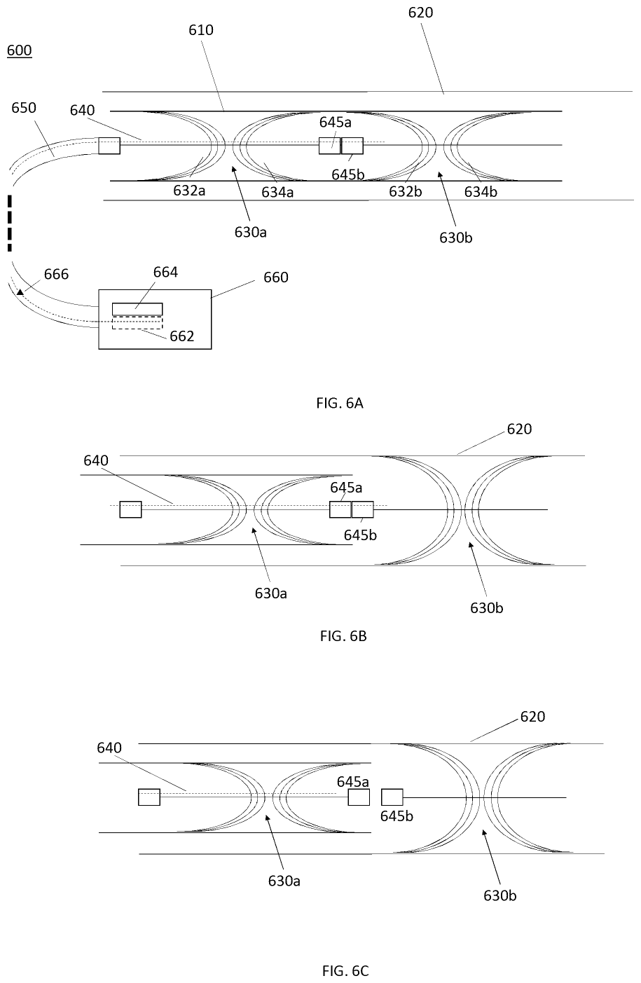

Once it is determined that a bristle section 305 is in the correct position within the bodily lumen, the elongate element 350 may then be retracted in the proximal direction by a predetermined distance relative to the delivery element until it is no longer received by the receiving element of the most distal bristle section. The delivery element which guides the bristle sections through the delivery catheter is held stationary relative to the bristle sections to prevent the bristle sections from being pulled proximally by the elongate element 350. Once the elongate element is no longer received by the receiving element of the bristle section, the bristle section is detached from the rest of the embolisation device 300 and is separately deployed within the bodily lumen to the remaining bristle sections, similar to the configuration shown in FIGS. 2 and 6B. The remaining bristle sections of the embolisation device 300 may be separately deployed within the bodily lumen in this manner.

Advantageously, the embolisation device 300 of FIG. 3 allows the bristle sections to be repositionable within a bodily lumen and to be separately deployed, which allows for higher controllability of deployment meaning the embolisation device 300 can be deployed in a greater variety of vessels. It is noted that the illustrated embodiment uses a single elongate element to connect a plurality of bristle sections. This may simplify the user interface to be used to actuate the elongate element. Alternatively, separate elongate elements may be used to connect one or more respective pairs of bristle sections, and may be actuated separately to detach the bristle sections in the manner described above in relation to FIGS. 3A and 3B.

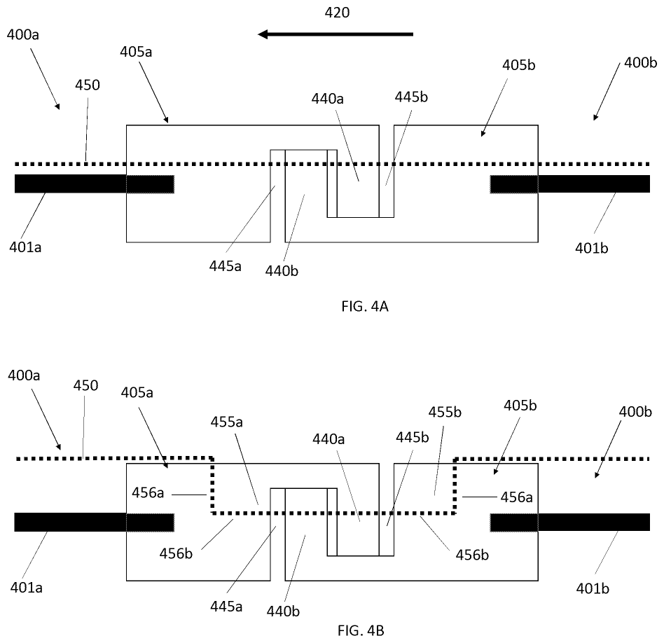

FIG. 4A shows a first embolisation bristle section 400a connected to a second embolisation bristle section 400b. Similar to the previously-disclosed examples, each bristle section 400a, 400b comprises one or more longitudinally connected bristle segments, each bristle segment comprising a core and a plurality of flexible bristles (not shown) extending radially outwardly from the core. As described in previous examples, the flexible bristles have a collapsed delivery configuration and an expanded deployed configuration in which the bristles extend generally radially outwardly from the core to anchor the device in a lumen. A proximal direction is indicated by arrow 420. The core 401a of the most distal bristle segment of bristle section 400a comprises a receiving element 405a mounted thereon. The core 401b of the most proximal bristle segment of bristle section 400b comprises a receiving element 405b mounted thereon. The receiving elements 400a, 400b may be mounted on the stem by adhesive, welding, crimping, or by any other manner of attachment.

The receiving elements 405a, 405b comprise respective interlocking features which can interlock with each other in a lateral direction (perpendicular to the longitudinal axis of the bristle sections, indicated by the arrow 420).

When the interlocking features are laterally interlocked as shown in FIG. 4A, relative displacement in the longitudinal direction between the bristle sections 400a and 400b is inhibited (although a limited range of longitudinal movement may be allowed, as illustrated by the longitudinal gaps between the receiving elements 405a and 405b). Furthermore, an elongate element 450 passes through the interlocking features in a longitudinal direction such that relative lateral movement between the bristle sections 400a and 400b is also inhibited. For example, the elongate element 450 may pass through holes in the interlocking features. As such, when the elongate element passes through the interlocking features, both lateral and longitudinal movement between the bristle sections is inhibited and the bristle sections are attached to one another.

In the example shown in FIG. 4A, the interlocking features 410a, 410b each comprise a lip 440a, 440b and a lateral recess 445a, 445b, wherein when interlocked the respective lips 440a, 440b are received by the lateral recess 445b, 445a of the other respective interlocking feature. In this example, the elongate element passes through both lips in a longitudinal direction to inhibit the relative lateral movement.

The receiving elements shown in FIG. 4A may be used as one or more of the receiving elements of the example in FIG. 3. Again, once it is determined that a bristle section 305 is in the correct position within the bodily lumen, the elongate element 350 may then be retracted in the proximal direction by a predetermined distance relative to the delivery element until it is no longer received by the receiving element of the most distal bristle section (for example, the receiving element 405b). Once the elongate element is no longer received by the receiving element, relative lateral movement between the most distal bristle section and the rest of the embolisation device is no longer restricted. Whilst longitudinal movement between the most distal bristle section and the rest of the embolisation device is initially inhibited, the most distal bristle section is in the expanded deployed configuration in the lumen. As such, the most distal bristle section still detaches from the rest of the embolisation device due to the lateral movement of the bristle section inside the lumen relative to the rest of the embolisation device, as the distal bristle section is anchored to the lumen and the rest of the embolisation device is still contained by the delivery catheter, and relative lateral movement between the delivery catheter and the lumen occurs. For example, as the delivery catheter is not anchored to the lumen, the distal tip of the delivery catheter naturally is not centred on the lumen, and is free to move laterally within the lumen relative to the longitudinal axis of the lumen. On the other hand, the most distal bristle section is anchored to the lumen which restricts lateral movement of the distal receiving element 405b. Accordingly, movement of the delivery catheter (and therefore the more distal non-deployed bristles sections) causes lateral movement which detaches the distal bristle section.

The bristle sections 400b and 400b may be configured to be one of many bristle sections making up an embolisation device. For example, a first bristle section 400b could be provided, and a second bristle section 400b may be connected to the first bristle section 400b as shown in FIG. 4A. Further bristle sections may be connected to the first or second bristle sections in a similar manner to create an embolisation device comprising a proximal bristle section, one or more intermediate bristle sections and a distal bristle section. One or both ends of the embolisation bristle sections 400b and 400b may additionally comprise a receiving element as described herein, depending whether the bristle sections 400b or 400b are configured to be an intermediate bristle section or a proximal or distal bristle section. The proximal bristle section may comprise a proximal connecting module for connecting to a delivery element, such as a male or female screw thread or a receiving element like those described herein.

FIG. 4B shows another pair of bristle sections according to another example. The example shown is similar to that shown in FIG. 4A and likewise numerals are used to denote likewise features. The example shown in FIG. 4B is the same as that shown in FIG. 4A, except the elongate element 450 extends through the receiving elements in a different manner. More specifically, bases 455a, 455b of receiving elements 405a, 405b each have a hole extending therethrough having a longitudinally extending section 456b and a laterally extending section 456a. The laterally extending sections 456a may extend radially inwards from any point on the surface of the bases 455a, 455b. The longitudinally extending sections 456b extend from the radial inward point of the corresponding laterally extending sections 456a towards the opposing receiving element. The elongate element 450 additionally passes through the longitudinally and laterally extending sections of the holes.

The receiving elements shown in FIG. 4B may be used as one or more of the receiving elements of the example shown in FIG. 3A. Its operation when used in an embolisation device like that shown in FIG. 3A is the same as described in relation to the receiving elements of FIG. 4A.

It is noted that, for example, a receiving element according to FIG. 4A could be interlocked with a receiving element according to FIG. 4B in an embolisation device.

The laterally extending section of the hole in the base of the receiving element increases the friction between the elongate element and the receiving element, which reduces the risk of the elongate element being extracted from the receiving element prematurely.

The receiving elements shown in FIGS. 4A and 4B may be used as one or of the receiving elements in any of the examples disclosed herein.

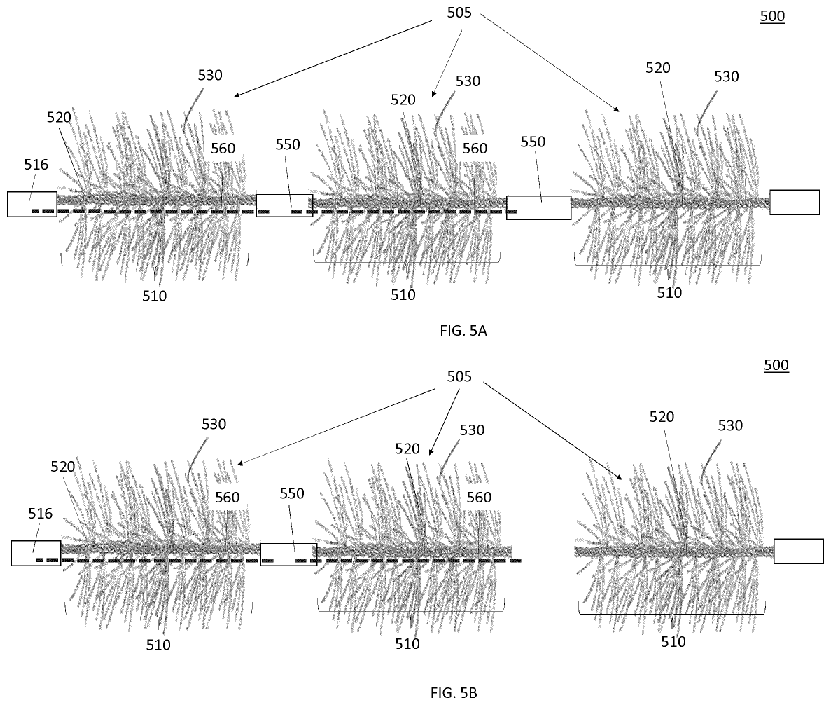

FIG. 5A shows an embolisation device 500 in an unconstrained configuration. The embolisation device 500 comprises three bristle sections 505. In other examples, the embolisation device may comprise two bristle sections or more than three bristle sections. Each bristle section comprises a single bristle segment 510. In other examples, each bristle section may comprise a different number of bristle segments. For example, one bristle section may comprise a single bristle segment and another may comprise two or more bristle segments. One or more bristle sections may comprise a flow restrictor mounted on the stem as disclosed previously. As in the example shown in FIG. 3A, each bristle segment 510 comprises a stem 520 and a plurality of bristles 530 having a collapsed delivery configuration and an expanded deployed configuration. Unlike the example shown in FIG. 3A, the embolisation device 500 comprises a plurality of electrolytic elements 550 attaching pairs of bristle sections 505. For example, the electrolytic element 550 may comprise an electrolytic material welding the stems of the respective bristle sections 505 together.

The embolisation device 500 further comprises a connecting module 516 at the proximal end of the embolisation device 500 for connecting to a delivery element. The connecting module may be any suitable connector such as a screw thread.

The electrolytic elements 550 are each electrically connected to the proximal end of the embolisation device (i.e. the end comprising the connecting module 516). For example, as shown in FIG. 5A, the embolisation device 500 may comprise electrical wires 560 extending between the ends of some of the bristle sections 505, such that the electrical connection is formed by the electrical wires 560 and electrolytic elements 550 to the proximal end of the device. The electrical wires 560 may be electrically isolated from the stems 520 and/or the bristles 530. This would inhibit corrosion of the stem and bristles in embodiments where they are made of materials which could corrode in the body lumen when an electrical current is applied, for example stainless steel or nitinol.

The electrolytic elements 550 each have a different disintegration energy. For example, the most distal electrolytic element 550 may be configured to disintegrate after a current of 0.01 to 0.1 mA at a voltage between 8 to 10V is applied for a period of time, for example 30 seconds. The next-most distal electrolytic element may be configured to disintegrate after a current between 0.5-1.10 mA at 5 to 7V is applied for a period of time, for example 30 seconds. The materials and/or geometries of each electrolytic element 550 can be selected in order to vary the disintegration energy of the electrolytic elements. For example, different materials may be selected for each electrolytic element 550 to achieve the different disintegration energies. Alternatively, a plurality of the electrolytic elements 550 may be made from the same material but may comprise different geometries to achieve different disintegration energies. For example, a first electrolytic element 550 may comprise a first volume of material attaching a pair of bristle sections 505, and a second electrolytic element 550 may comprise a second volume of the same material attaching another pair of bristle sections 505. The first and second volumes may be varied in order to select the desired disintegration energy, which can be measured through routine analysis. A greater volume of material forming an electrolytic element 550 generally corresponds to a higher disintegration energy. Alternatively, the electrolytic elements 550 may comprise the same material and have the same external geometry, but the internal porosity may be varied to vary the disintegration energy of the electrolytic elements 550. In yet further embodiments, the electrolytic elements may comprise the same material and have the same external geometry, but may be coated or partially coated by materials to partially increase or decrease the resistance of the material of the electrolytic element 550 from corroding. The level of coating may be varied between the electrolytic elements to vary the disintegration energies.

Suitable materials for the electrolytic elements 550 include stainless steels, Ti or TiNi, cobalt alloys, noble or non-noble metals. Suitable materials for the coating of the electrolytic elements 550 include insulating materials or zinc or tin or alloys thereof.

FIG. 5B shows a configuration of the device 500 after the most distal electrolytic element 550 has disintegrated. As a result, the most distal bristle section 505 is separated from the rest of the device 500. Accordingly, each bristle section 505 may be independently deployed from the device 500 as described below.

Similar to the system shown in FIG. 2, the embolisation device 500 may be placed in a delivery catheter for delivery to a target site in a bodily lumen. A delivery element comprising an electrical wire may be connected to the connecting module 516 at the proximal end of the embolisation device 500, for example via a screw thread mechanism or other. When the delivery element is connected to the connecting module 516, an electrical connection is formed between the delivery element and the electrolytic elements 550 (either via the connecting module being made of metal, or directly connecting to the most proximal electrical wire of the most proximal section 505). The delivery element, connected to the proximal end of the embolisation device 500, is used to guide the bristle sections 505 through the delivery catheter in a proximal direction towards a target site within the bodily lumen.

The bristle sections may then be deployed from the distal end of the delivery catheter to a target site of the bodily lumen. When a bristle section 505 exits the distal end of the delivery catheter (i.e. the most distal bristle section), the bristle section 505 moves from a collapsed delivery configuration within the delivery catheter to an expanded deployed configuration in which the bristles of the bristle section (i.e. the bristles 530 of the one or more bristle segments 510) engage the walls of the bodily lumen to anchor the bristle section 505 to the lumen. When the bristle section 305 is positioned within the bodily lumen in the expanded deployed configuration (i.e. when it has exited the delivery catheter), it is initially still attached to the next most proximal bristle section via electrolytic element 550, and relative longitudinal and lateral movement between these bristle sections is still inhibited. As such, if it is determined that the most distal bristle section is incorrectly positioned, the delivery element may be moved in a proximal direction relative to the delivery catheter so that the bristle sections 505 all move proximally (due to the chain of connections between the delivery element and the linearly connected bristle sections), such that the distal bristle section in the bodily lumen is recaptured by the delivery catheter. The bristle section may then be delivered from the delivery catheter to a new position within bodily lumen.

Once it is determined that a bristle section 505 is in the correct position within the bodily lumen, a positive electric current may be applied to the device 500 through the delivery element until the electrolytic element 550 of the most distal bristle section disintegrates. Once disintegrated, the most distal bristle section 505 is detached from the rest of the embolisation device 500 and is deployed within the bodily lumen, similar to the configuration shown in FIGS. 2 and 6B. The remaining bristle sections of the embolisation device 500 may be separately deployed within the bodily lumen in this manner.

FIG. 6A shows an embolisation delivery system 600 for delivering an embolisation device. In the example shown in FIG. 6A, a delivery catheter 610 of the system 600 is shown inserted in a bodily lumen 620. An embolisation device comprising two bristle sections 630a, 630b is shown in the collapsed delivery configuration inside the delivery catheter 610. Each bristle section comprises a proximal bristle segment 632a, 632b, and a distal bristle segment 634a, 634b. The proximal bristle segments 632a, 632b are all configured to point longitudinally in a proximal direction whereas the distal bristle segments 634a, 634b are all configured to point longitudinally in a distal direction. The bristle segments are shown as being formed on the same stem, whereas in other examples the bristle segments may be joined by a non-articulating or an articulating joint as disclosed above, for example. The bristle sections 630a, 630b are connected to one another via an elongate element 640 slidably received by respective receiving elements 645a, 645b (in other examples other connecting mechanisms as disclosed herein are used to connect the bristle sections).

A proximal end of the embolisation device is connected to a delivery element 650, such as by a screw thread or via any other releasable connecting mechanism. The elongate element 640 terminates at the opposite end at deployment system 660 comprising an actuator and a user interface. For example, the actuator may be a spool 662 housed within the deployment system 660 onto which the elongate element 640 is wound, and the user interface may be a roller wheel for allowing the user to roll the spool.

Using the system 600, a distal end of the delivery catheter 610 is positioned at a target location within the bodily lumen. When the correct position is reached, the delivery element 650 is moved in a distal direction relative to the delivery catheter 610 so that the bristle section 630b is delivered to the bodily lumen 620. Such a configuration is shown in FIG. 6B (with the delivery element 650 and deployment system 660 omitted for simplicity). In this configuration, the bristle section 630b is in an expanded deployed configuration within the bodily lumen 620. However, the bristle sections 630a and 630b are still attached due to the elongate element 640 being received by both receiving elements 645a and 645b. Therefore, if it is determined that the bristle section 630b has been incorrectly deployed, then the delivery element can be retracted in a proximal direction relative to the delivery catheter 610 and the bristle section 630b can be recaptured and redeployed within the lumen.

Once it is determined that the bristle section 630b is correctly deployed, the elongate element 640 can be retracted by the required predetermined distance via the actuator (e.g. spool 662) using the user interface (e.g. wheel 664) so that it is no longer received by the receiving element 645b. This configuration is shown in FIG. 6C.

When the system 600 is used for embolisation devices comprising one or more elongate elements slidable between the first and second configurations, the user interface comprises an actuator, and more specifically a retracting element (such as a rotatable wheel) which is used to slide the connecting mechanism(s) between the first and second configurations. The delivery system may comprise a feedback mechanism for indicating to the user that a particular detachment has occurred (for example providing a visual indication or a sound, or the elongate element 640 may comprise suitable markers 666 along its length as indicators that it has been sufficiently retracted to detach a bristle section for each bristle section).

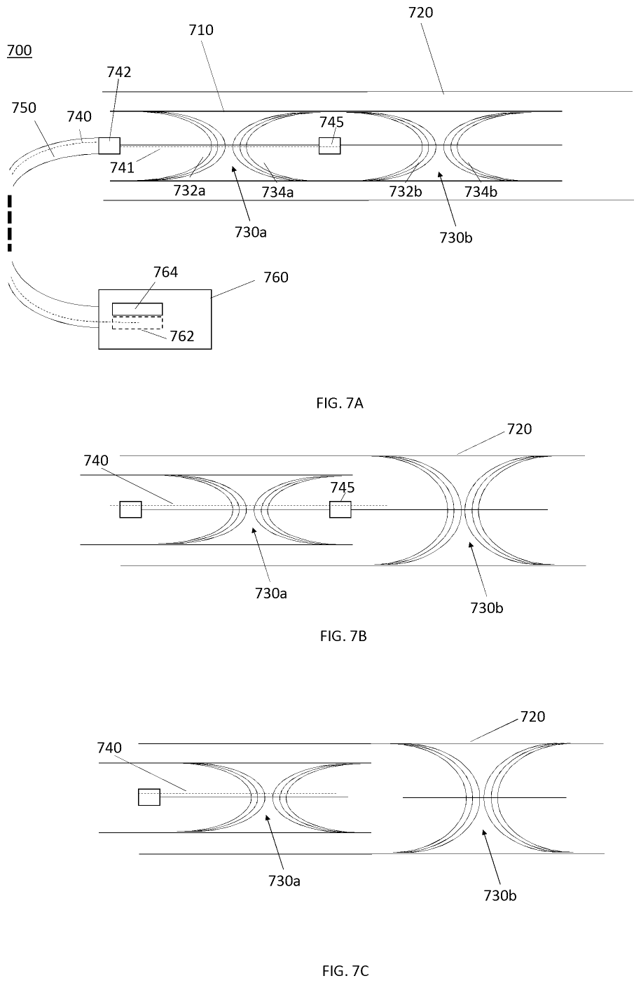

FIG. 7A shows an embolisation delivery system 700 for delivering an embolisation device. In the example shown in FIG. 7A, a delivery catheter 710 of the system 700 is shown inserted in a bodily lumen 720. An embolisation device comprising two bristle sections 730a, 730b is shown in the collapsed delivery configuration inside the delivery catheter 710. Each bristle section comprises a proximal bristle segment 732a, 732b, and a distal bristle segment 734a, 734b. The proximal bristle segments 732a, 732b are all configured to point longitudinally in a proximal direction whereas the distal bristle segments 734a, 734b are all configured to point longitudinally in a distal direction. The bristle segments are shown as being formed on the same stem, whereas in other examples the bristle segments may be joined by a non-articulating or an articulating joint as disclosed above, for example. The bristle sections 730a, 730b are connected to one another via an electrolytic element 745.

A proximal end of the embolisation device is connected to a delivery element 750, such as by a screw thread or via any other releasable connecting mechanism. The delivery element 750 comprises an electrical wire 740. The electrical wire 740 forms an electrical connection with the electrolytic element 745, either directly with electrical wire 741 on the proximal bristle section 730a, or via the module 742 attaching the delivery element 750 to the embolisation device. The delivery element 750 and electrical wire 740 terminate at the opposite end at deployment system 760 comprising an actuator 762 and a user interface 764. For example, the actuator may be a positive current or voltage source electrically connected to the electrical wire 740 and the user interface 764 may be any suitable user interface for actuating the current source. The negative pole of the electrical supply of the actuator 762 may be placed in electrical contact with the skin to complete the electrical circuit.

Using the system 700, a distal end of the delivery catheter 710 is positioned at a target location within the bodily lumen. When the correct position is reached, the delivery element 750 is moved in a distal direction relative to the delivery catheter 710 so that the bristle section 730b is delivered to the bodily lumen 720. Such a configuration is shown in FIG. 7B (with the delivery element 750 and deployment system 760 omitted for simplicity). In this configuration, the bristle section 730b is in an expanded deployed configuration within the bodily lumen 720. However, the bristle sections 730a and 730b are still attached via the electrolytic element 745. Therefore, if it is determined that the bristle section 730b has been incorrectly deployed, then the delivery element can be retracted in a proximal direction relative to the delivery catheter 710 and the bristle section 730b can be recaptured and redeployed within the lumen.

Once it is determined that the bristle section 730b is correctly deployed, a positive electric current can be applied to the electrolytic element 745 via the actuator 762 using the user interface 764 so that it disintegrates from the ionic fluid in the body lumen and the bristle sections are no longer attached. This configuration is shown in FIG. 7C.

When the system 700 is used for embolisation devices comprising one or more electrolytic elements, the actuator comprises a positive current or voltage source for applying the necessary voltage to the electrolytic element, which is actuatable by a user (for example via a switch or button). The user interface may provide the user with means to select the current, voltage and duration of application. (i.e. the voltage source may be variable and selectable by the user).

Also disclosed herein is a method of manufacturing an embolisation device for promoting clot formation in a body lumen comprising:
providing a plurality of bristle sections each comprising one or more bristle segments, each bristle segment comprising a core and a plurality of flexible bristles extending radially outwardly from the core, the flexible bristles having a collapsed delivery configuration and an expanded deployed configuration in which the bristles extend at least radially outwardly from the core to anchor the bristle section in a lumen; and
linearly connecting adjacent pairs of the bristle sections via one or more connecting mechanisms, each connecting mechanism being operable to change from a first configuration attaching a pair of adjacent sections, to a second configuration detaching the pair of adjacent sections, when the bristle segments are in the expanded deployed configuration.

Also disclosed herein is a method of manufacturing an embolisation bristle section for promoting clot formation in a body lumen, comprising:
providing one or more bristle segments, each bristle segment comprising a core and a plurality of flexible bristles extending radially outwardly from the core, the flexible bristles having a collapsed delivery configuration and an expanded deployed configuration in which the bristles extend at least radially outwardly from the core to anchor the device in a lumen; and
providing a receiving element configured to slidably receive an elongate element for connecting the bristle section to an adjacent bristle section, wherein the receiving element is configured to allow the elongate element to slide between a first configuration attaching the bristle section and the adjacent bristle section, and a second configuration detaching the bristle segment from the adjacent bristle section.

The embolisation devices disclosed above allow a user to create a custom embolisation device for a given vessel. In particular, a user may construct an embolisation device which comprises a number of different bristle sections which are specifically adapted to the vessel. For instance, an embolisation device may be constructed which has a bristle section comprising one or more bristle segments of a first diameter, and a bristle section comprising one or more bristle segments of a second diameter. The user is able to connect the desired bristle sections and is able to controllably deploy each bristle section separately within a bodily lumen, in a single operation.

For embodiments wherein the bristle sections are connected via an elongate element and receiving elements, the embolisation device may be provided as a kit of parts. For example, a kit of parts may be provided which includes a large variety of bristle sections such that a custom embolisation device may be constructed by a user depending on the vasculature where the sections are to be implanted.

All of the above are fully within the scope of the present disclosure, and are considered to form the basis for alternative embodiments in which one or more combinations of the above described features are applied, without limitation to the specific combination disclosed above.

In light of this, there will be many alternatives which implement the teaching of the present disclosure. It is expected that one skilled in the art will be able to modify and adapt the above disclosure to suit its own circumstances and requirements within the scope of the present disclosure, while retaining some or all technical effects of the same, either disclosed or derivable from the above, in light of his common general knowledge in this art. All such equivalents, modifications or adaptations fall within the scope of the present disclosure.

1. An embolisation device for promoting clot formation in a body lumen comprising two or more linearly connected sections, each section comprising one or more bristle segments comprising a core and a plurality of flexible bristles extending at least radially outwardly from the core, the flexible bristles having a collapsed delivery configuration and an expanded deployed configuration in which the bristles extend at least radially outwardly from the core to anchor the bristle segment in a bodily lumen, wherein:
each pair of adjacent sections are connected via a respective connecting mechanism; and
when the bristle segments are in the expanded deployed configuration, each respective connecting mechanism is selectively changeable from a first configuration attaching the pair of adjacent sections, to a second configuration detaching the pair of adjacent sections.

2. The embolisation device of claim 1, wherein at least one connecting mechanism is movable from its first configuration to its second configuration.

3. The embolisation device of claim 1 or 2, wherein at least one connecting mechanism is formed by an elongate element slidably received by respective receiving elements of respective adjacent sections, such that the elongate element is slidable by a predetermined distance between the first and second configurations, and wherein in the second configuration the elongate element detaches from one of the receiving elements.

4. The embolisation device of claim 3, wherein the embolisation device has a longitudinal axis and the receiving elements each comprise respective interlocking features, wherein in the first configuration the respective interlocking features interlock with one another in a lateral direction to inhibit relative displacement in the longitudinal direction between the pair of adjacent sections, and the elongate element passes through both interlocking features in a longitudinal direction to inhibit relative lateral movement between the pair of adjacent sections.

5. The embolisation device of claim 4, wherein the respective interlocking features each comprise a lip and a lateral recess, wherein in the first configuration the respective lips are received by the lateral recess of the other respective interlocking feature, and the elongate element passes through both lips in a longitudinal direction to inhibit relative lateral movement between the pair of adjacent sections.

6. The embolisation device of claim 5, wherein a first of the pair of receiving elements comprises a base longitudinally separated from the lip by the recess, the base having a hole extending therethrough having a longitudinally extending section and a laterally extending section, wherein in the first configuration the elongate element passes through the lip of the first receiving element, the lip of the other receiving element, the longitudinally extending section of the hole and the laterally extending section of the hole.

7. The embolisation device of any of claims 3 to 6, wherein at least two of the connecting mechanisms are formed by the same elongate element received by a plurality of pairs of receiving elements such that the elongate element is slidable by a plurality of predetermined distances to detach a pair of sections at each predetermined distance.

8. The embolisation device of claim 1, wherein at least one connecting mechanism is formed by an electrolytic element attaching a respective pair of adjacent sections, the electrolytic element being electrically connected to a proximal end of the embolisation device, and operable to disintegrate by electrolysis in the body lumen to detach the respective pair of adjacent sections by applying an electric current to the electrolytic element, at a current amplitude, a voltage and for a duration of time such that the electrical energy supplied to the electrolytic element is above a disintegration energy of the electrolytic element.

9. The embolisation device of claim 8, wherein the electrical connection between the proximal end of the embolisation device to the electrolytic element is electrically isolated from the cores and/or bristles of the embolisation device.

10. The embolisation device of claim 8 or 9, wherein at least two connecting mechanisms are each formed by an electrolytic element electrically connected to the proximal end of the embolisation device, and wherein the disintegration energy of each electrolytic element is different.

11. An embolisation bristle section for promoting clot formation in a body lumen comprising one or more linearly connected bristle segments, each bristle segment comprising a core and a plurality of flexible bristles extending at least radially outwardly from the core, the flexible bristles having a collapsed delivery configuration and an expanded deployed configuration in which the bristles extend at least radially outwardly from the core to anchor the device in a lumen, wherein:
the bristle section further comprises a receiving element configured to slidably receive a connecting mechanism for connecting the bristle section to an adjacent bristle section, wherein the receiving element is configured to allow the connecting mechanism to slide between a first configuration for attaching the bristle section and the adjacent bristle section, and a second configuration for detaching the bristle segment from the adjacent bristle section.

12. An embolisation delivery system for delivering an embolisation device according to any of claims 1 to 10, comprising:
a delivery catheter for containing the embolisation device and having a distal delivery end;
a delivery element for delivering the embolisation device from the distal end of the delivery catheter;
an actuator for changing the one or more connecting mechanisms from the first configuration to the second configuration; and
one or more user interfaces for operating the delivery element and the actuator.

13. The embolisation delivery system of claim 12, wherein when one or more of the connecting mechanisms comprise one or more elongate elements slidable between first and second configurations, the actuator comprises a retracting element operable by a user to slide the one or more connecting mechanisms from the first to the second configuration; and/or
when one or more of the connecting mechanisms comprise one or more electrolytic elements changeable from the first configurations to the second configurations by application of a positive current to the one or more electrolytic elements, the actuator is operable by a user to apply a positive current to the one or more connecting mechanisms for detachment.

14. The embolisation delivery system of claim 13, wherein when one or more of the connecting mechanisms are elongate elements slidable between the first and second configurations, the delivery system comprises a feedback mechanism for indicating to the user that the one or more connecting mechanisms have slid a sufficient distance to cause detachment.

15. The embolisation delivery system of claim 13 or 14, wherein when one or more of the connecting mechanisms are electrolytic elements, the actuator is operable to apply a variable positive current and voltage selectable by the user via the user interface.

16. A method of manufacturing an embolisation device for promoting clot formation in a body lumen comprising:
providing a plurality of bristle sections each comprising one or more bristle segments, each bristle segment comprising a core and a plurality of flexible bristles extending at least radially outwardly from the core, the flexible bristles having a collapsed delivery configuration and an expanded deployed configuration in which the bristles extend at least radially outwardly from the core to anchor the bristle section in a lumen;
linearly connecting adjacent pairs of the bristle sections via one or more connecting mechanisms, each connecting mechanism being operable to change from a first configuration attaching a pair of adjacent sections, to a second configuration detaching the pair of adjacent sections, when the bristle segments are in the expanded deployed configuration.

17. A method of manufacturing an embolisation bristle section for promoting clot formation in a body lumen, comprising:
providing one or more bristle segments, each bristle segment comprising a core and a plurality of flexible bristles extending at least radially outwardly from the core, the flexible bristles having a collapsed delivery configuration and an expanded deployed configuration in which the bristles extend at least radially outwardly from the core to anchor the device in a lumen; and
providing a receiving element configured to slidably receive an elongate element for connecting the bristle section to an adjacent bristle section, wherein the receiving element is configured to allow the elongate element to slide between a first configuration attaching the bristle section and the adjacent bristle section, and a second configuration detaching the bristle segment from the adjacent bristle section.

18. A kit of parts for making an embolisation device, comprising:
two or more sections, each section comprising:
one or more bristle segments comprising a core and a plurality of flexible bristles extending at least radially outwardly from the core, the flexible bristles having a collapsed delivery configuration and an expanded deployed configuration in which the bristles extend at least radially outwardly from the core to anchor the bristle segment in a lumen; and
one or more receiving elements configured to slidably receive an elongate element for connecting the bristle section to an adjacent bristle section, wherein the receiving element is configured to allow the elongate element to slide between a first configuration attaching the bristle section and the adjacent bristle section, and a second configuration detaching the bristle segment from the adjacent bristle section;
the kit of parts further comprising one or more elongate elements for connecting the bristle sections.
